(19) United States
(12) Patent Application Publication
(10) Pub. No.: US 2024/0407852 A1
(43) Pub. Date: Dec. 12, 2024

(54) Assembly And Method For Mounting A Tracker To Bone

(71) Applicant: MAKO Surgical Corp., Weston, FL (US)

(72) Inventors: Timothy Wade Perez, Plantation, FL (US); Adam L. Nagy, Coconut Creek, FL (US); Anthony Andrew Laviano, Hollywood, FL (US); Nicolas Perilla, Fort Lauderdale, FL (US); Matthew Thompson, Woodbridge, CT (US)

(73) Assignee: MAKO Surgical Corp., Weston, FL (US)

(21) Appl. No.: 18/734,863

(22) Filed: Jun. 5, 2024

Related U.S. Application Data
(60) Provisional application No. 63/472,138, filed on Jun. 9, 2023.

(51) Int. Cl. A61B 34/20; A61B 90/00

(52) U.S. Cl. CPC A61B 34/20; A61B 90/39; A61B 2034/2072; A61B 2090/3916

(57) ABSTRACT

A tracker mount includes a fastener assembly, a sleeve assembly to be disposed over the fastener assembly, and a tracker interface coupled to the sleeve assembly and configured to removably attach to a tracker. The fastener assembly includes a fastener adapted to be attached to a bone and a first keying feature. The sleeve assembly includes a sleeve, which includes a body defining a channel including a second keying feature to interface with the first keying feature to prevent rotation of the sleeve relative to the fastener assembly. The sleeve assembly includes an engagement feature at a distal part of the sleeve to engage a surface of the bone.

### CROSS REFERENCE TO RELATED APPLICATIONS

This application claims benefit from U.S. Provisional Patent Application No. 63/472,138 filed 9 Jun. 2023, the contents of which are hereby expressly incorporated by reference thereto in its entirety for all purposes.

### BACKGROUND

Surgical navigation systems assist users in locating surgical objects in the operating room. The navigation system includes a localizer to determine the position and/or orientation of a surgical object using a tracking device attached to the surgical object. The surgical object is often an instrument, device, or an anatomic object, such as bone.

The tracking device can be attached to a bone using a bone mount. Conventional bone mounts are susceptible to several issues. For example, conventional bone mounts typically require multiple fasteners to be inserted into the bone, which can cause damage to the bone. Furthermore, conventional bone mounts are susceptible to rotational instability, thereby causing the bone mount, and attached tracking device, to become lose. Such instability compromises the localizer's ability to accurately track the bone. Conventional bone mounts also are disposable and intended for single use. These single-use bone mounts lack durability, which can cause problems with the stability of the design. Moreover, conventional bone mounts are not ergonomically optimized. For example, certain bone mounts require the user to blindly drive a fastener through a sleeve that obstructs the visibility of the fastener to the user. Additionally, conventional bone mounts often exhibit a large surface footprint which may cause interference or collisions at the surgical site.

### SUMMARY

This Summary introduces a selection of concepts in a simplified form that are further described below in the Detailed Description. This Summary is not intended to limit the scope of the claimed subject matter nor identify key features or essential features of the claimed subject matter.

According to a first aspect, a tracker mount is provided. The tracker mount includes: a fastener assembly comprising: a fastener adapted to be attached to a bone; and a first keying feature; and a sleeve assembly adapted to be disposed over the fastener assembly and comprising: a sleeve comprising: a body defining a channel, wherein the channel includes a second keying feature to interface with the first keying feature to prevent rotation of the sleeve relative to the fastener assembly; and an engagement feature at a distal part of the sleeve to engage a surface of the bone; and a sleeve assembly driver at least partially disposed within the channel, the sleeve assembly driver being adapted to be rotated to couple to the fastener assembly and move the engagement feature into the bone; and a tracker interface coupled to the sleeve assembly and configured to removably attach to a tracker.

According to a second aspect, a method of mounting a tracker mount to a bone, the tracker mount including a fastener assembly, a sleeve assembly, and a tracker interface coupled to the sleeve assembly, the fastener assembly including a fastener and a first keying feature, the sleeve assembly including a sleeve and a sleeve assembly driver, the sleeve including an engagement feature at a distal part of the sleeve and a body defining a channel including a second keying feature, the method comprising steps of: attaching the fastener of the fastener assembly to the bone; after attaching the fastener, disposing the sleeve assembly over the fastener assembly such that the second keying feature interfaces with the first keying feature for preventing rotation of the sleeve relative to the fastener assembly; and rotating the sleeve assembly driver to couple the sleeve assembly driver to the fastener assembly for moving the engagement feature into the bone.

According to a third aspect, a bone mount is provided. The tracker mount includes a fastener assembly, a sleeve assembly to be disposed over the fastener assembly, and a tracker interface coupled to the sleeve assembly and configured to removably attach to a tracker. The fastener assembly includes a first keying feature and a fastener adapted to be attached to a bone. The sleeve assembly includes a sleeve, which includes a body defining a channel including a second keying feature to interface with the first keying feature to prevent rotation of the sleeve relative to the fastener assembly. The sleeve assembly includes an engagement feature at a distal part of the sleeve to engage a surface of the bone.

Any of the above aspects can be utilized individually, or in combination.

Any of the above aspects can be utilized with any of the following optional implementations.

In one implementation, the fastener assembly further comprises a fastener assembly driver coupled to the fastener and adapted to receive force/torque to attach the fastener to the bone. In one implementation, the fastener assembly driver of the fastener assembly is permanently fixed to the fastener. In one implementation the sleeve assembly driver is configured to be rotated to couple to the fastener assembly driver of the fastener assembly. In one implementation, the sleeve assembly driver defines a second channel configured to receive the fastener assembly driver, and wherein the fastener assembly driver further comprises first threads and the sleeve assembly driver further comprises second threads located in the second channel to interface with the first threads in response to rotation of the sleeve assembly driver.

In one implementation, the fastener assembly further comprises a stop located above the fastener for limiting driving of the fastener beyond a predetermined distance. In one implementation, the stop is configured to contact the bone to limit driving of the fastener beyond the predetermined distance. The fastener can be any type of fastener, such as a screw, poly-axial screw, bone-pin, or nail, a bone claw, a bone clamp, or the like.

In one implementation, the sleeve assembly is adapted to be disposed over the fastener assembly after the fastener of the fastener assembly is attached to the bone.

In one implementation, the sleeve assembly driver includes an abutment configured to contact the sleeve such that rotation of the sleeve assembly driver causes the abutment to move the sleeve to move the engagement feature into the bone. In one implementation, the body of the sleeve includes a projection and the sleeve assembly driver includes a receiving portion shaped to receive the projection to axially lock the body of the sleeve to the sleeve assembly driver such that, when the receiving portion receives the projection, rotation of the sleeve assembly driver moves the sleeve to move the engagement feature into the bone.

In one implementation, the first keying feature of the fastener assembly includes external polygonal shaped walls, and the second keying feature of the sleeve assembly includes internal polygonal shaped walls. In one implementation, the first keying feature is located between the fastener and the fastener assembly driver. In one implementation, the channel of the sleeve includes a first end and a second end, wherein the sleeve assembly driver extends through the first end, and wherein the second keying feature is located at the second end.

In one implementation, the engagement features comprise one or more teeth integrally formed with the sleeve and configured to engage the surface of the bone.

In one implementation, a support arm is coupled to the sleeve assembly and the tracker interface is coupled to the support arm.

In one implementation, after attaching the fastener assembly, the sleeve assembly is disposed over the fastener assembly such that the second keying feature interfaces with the first keying feature to prevent rotation of the sleeve relative to the fastener assembly.

In one implementation, removably attaching a tracker to the tracker interface occurs before, during, or after rotating the sleeve assembly driver to couple the sleeve assembly driver to the fastener assembly and moving the engagement features into the bone.

### BRIEF DESCRIPTION OF THE DRAWINGS

Advantages of the present invention will be readily appreciated as the same becomes better understood by reference to the following detailed description when considered in connection with the accompanying drawings wherein:

FIG. 1 is a perspective view of a tracker mount mounted to a femur bone, according to one implementation.

FIG. 2 is a front view of the tracker mount, according to one implementation.

FIG. 3 is a front view of one implementation of a fastener assembly of the tracker mount.

FIG. 4 is a front view of one implementation of a sleeve assembly of the tracker mount.

FIG. 5 is a cross-sectional view of the sleeve assembly, according to one implementation.

FIG. 6 is a cross-sectional view of the sleeve assembly coupled to the fastener assembly, according to one implementation wherein the driver of the sleeve assembly and the sleeve body are axially constrained with one another.

FIG. 7 is a cross-sectional view of the sleeve assembly coupled to the fastener assembly, according to another implementation wherein the driver of the sleeve assembly is configured to axially move freely within the sleeve body.

FIG. 8 is a cross-sectional view of the sleeve assembly coupled to the fastener assembly, according to another implementation wherein the driver of the sleeve assembly is configured to axially move within a range of motion within the sleeve body.

FIG. 9 is a flowchart of one implementation of a method of mounting the tracker mount to a bone.

FIGS. 10A-10F illustrate example steps of mounting the tracker mount to a bone.

### DETAILED DESCRIPTION

### I. Overview

Referring to the Figures, wherein like numerals indicate like or corresponding parts throughout the several views, an example of a tracker mount 10 is shown throughout.

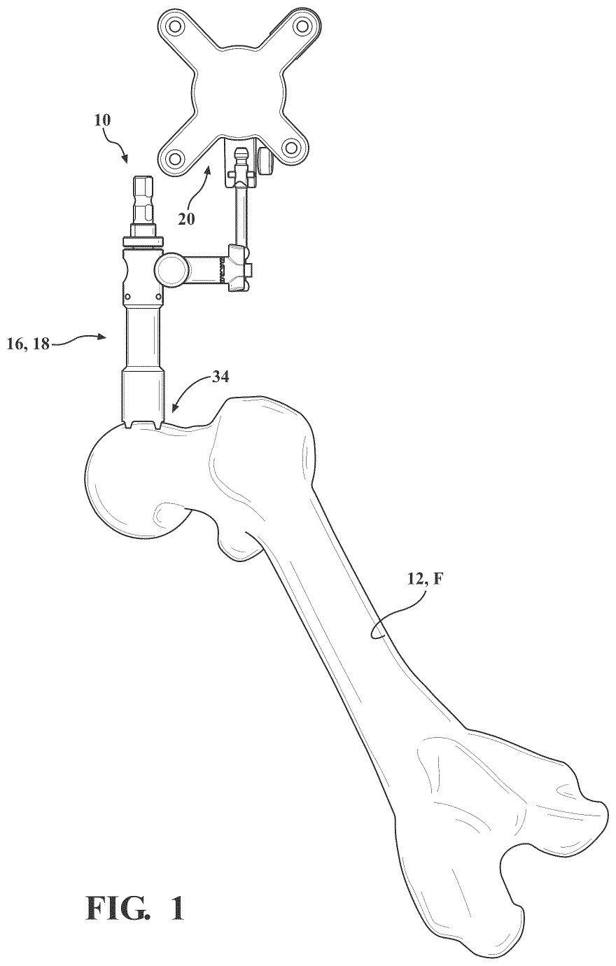

As shown in FIG. 1, the tracker mount 10 is configured to be mounted to an anatomy 12 of a patient, such that a surgical system may track a tracker 48 attached to the tracker mount 10 to determine a position and/or orientation of the anatomy 12. In the instance of FIG. 1, the anatomy 12 includes a femur (F) of the patient and the tracker mount 10 is mounted to the femur (F) such that a surgical system may track the tracker mount 10 to determine a position and/or orientation of the femur (F). The tracker mount 10 can be mounted to a proximal portion of the femur (F), such as the femoral head, as shown. Alternatively, the tracker mount can be mounted to the greater trochanter, femoral shaft, or femoral neck. In other instances, the anatomy 12 may include any other bone or soft tissue of the patient, and the tracker mount 10 may be mounted to any other bone or soft tissue of the patient. For example, the tracker mount 10 can be coupled to a humeral bone, such as at the humeral head.

The surgical system may be any surgical system configured to track the tracker 48 attached to the tracker mount 10 and perform a surgical procedure on the anatomy 12. For example, the surgical system may include a surgical navigation system configured to track the tracker 48. The surgical navigation system can track the tracker 48 using any type of tracking modality, and the tracker 48 can be configured to be tracked using any modality, such as optical (passive or active), machine vision (e.g., pattern or shape recognition), electromagnetic, radio frequency, ultrasound tracking, and the like. Additionally, the surgical system may include a robotic surgical system configured to perform a surgical procedure. Examples of navigation systems, tools, and robotic systems which can be utilized with the tracker mount described herein can be like that described in United States Patent App. Pub. No. US 2022/0218422, entitled “Surgical Systems and Methods for Guiding Robotic Manipulators”, and U.S. Pat. No. 11,478,362, entitled “Robotic Surgery System for Augmented Hip Arthroplasty Procedures”, each of which is hereby incorporated by reference in their entirety.

In one example, the surgical procedure performed by the surgical system is a total hip replacement surgery. Alternatively, the surgical procedure may be a partial hip replacement surgery, or a total or partial knee replacement procedure. In other examples, the surgical procedure may be a shoulder arthroplasty surgery, such as anatomical shoulder or reverse shoulder arthroplasty. In other examples, the surgical procedure may be a revision surgery for any of the described procedures. Alternatively, or additionally, the surgical procedure may involve tissue removal or treatment. Treatment may include cutting, coagulating, lesioning the tissue, treatment in place of tissue, or the like. In one example, the surgical system is designed to cut away material to be replaced by surgical implants, such as acetabular implants, proximal femur implants, glenoid implants, humeral implants, hip, and knee implants, including unicompartmental, bicompartmental, multicompartmental, or total knee implants, and the like. The surgical system may be used to perform other procedures, surgical or non-surgical, or may be used in industrial applications or other applications where robotic systems are utilized.

### II. Tracker Mount Structure

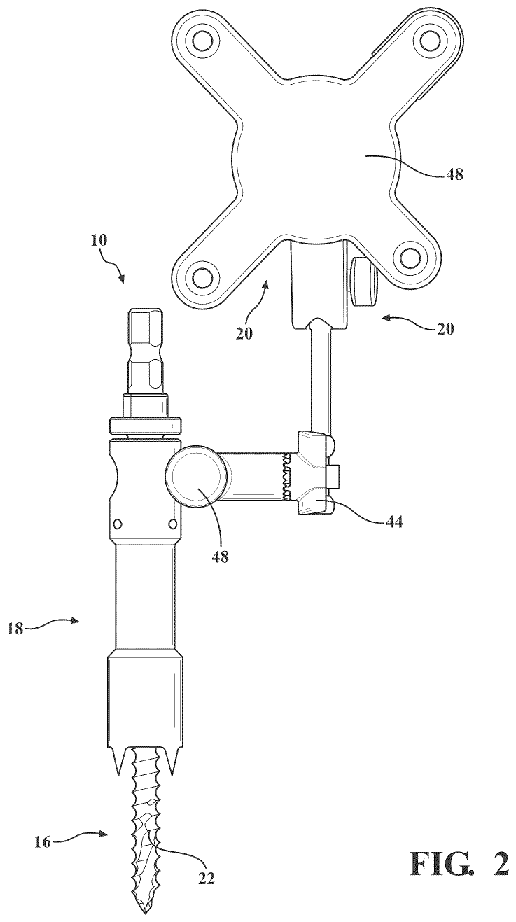

The tracker mount 10 is further shown in FIG. 2. As shown, the tracker mount 10 includes a fastener assembly 16, a sleeve assembly 18 adapted to be coupled to, or disposed over, the fastener assembly 16, and a tracker interface 20 coupled to the sleeve assembly 18. The fastener assembly 16 is configured to be attached to the bone 12. After the fastener assembly 16 is attached to the bone 12, the sleeve assembly 18 couples to the fastener assembly 16 and engages the bone surface to provide a stable mount for the tracker 48 relative to the bone 12.

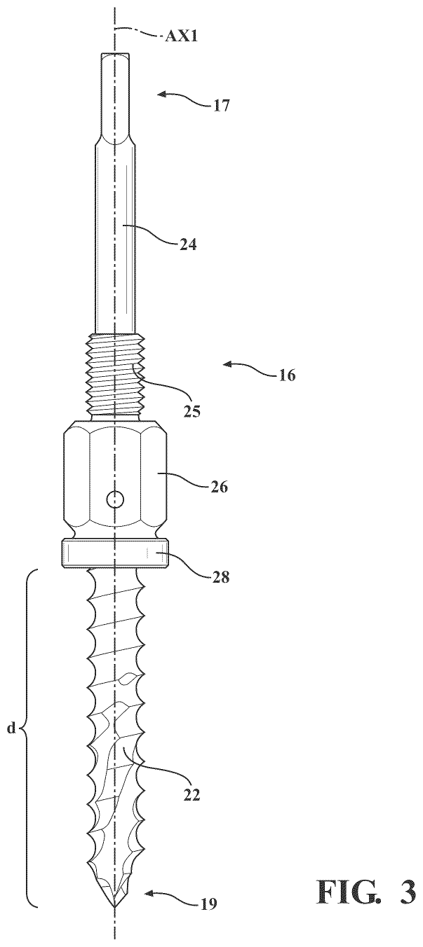

One example of the fastener assembly 16 is further shown in FIG. 3. As shown, the fastener assembly 16 extends between a proximal part 17 and a distal part 19 along an axis AX1. The fastener assembly 16 includes a fastener 22 located at the distal part 19. The fastener 22 is adapted to be attached to the bone, such as the femur (F), as shown in FIG. 1. The fastener 22 can be a screw, a bone pin, a nail, a bone clamp, or a bone claw. Additionally or alternatively, the fastener 22 may comprise an assembly of a bone plate that is fastened to the bone using one or more fasteners. The fastener assembly 16 may be rigid such that the components are fixed relative to each other. The fastener assembly 16 may include parts that are movable relative to one another. The fastener assembly 16 may be entirely monolithic or a unitary structure. Alternatively, the distal part 19 may be separable from the proximal part 17. In another example, one or more components of the proximal part 17, i.e. the fastener assembly driver 24, 25, 26, may be separable from the distal part 19 and, optionally, disposable or single-use components. Additionally, it should be noted that a size and/or a shape of the fastener 22 may selected to allow for other components of the tracker mount 10 to interface with the fastener assembly 16 as described herein. For example, in instances where the fastener 22 is a bone clamp, the bone clamp may include a size and/or shape to allow the sleeve assembly 18 to be disposed over the fastener assembly 16.

The fastener assembly 16 also includes a fastener assembly driver 24 coupled to the fastener 22 and located at the proximal part 17. The fastener assembly driver 24 is configured to receive force/torque to attach the fastener 22 to the bone. When the fastener 22 is a screw, the fastener assembly driver 24 is adapted to be rotated to rotatably attach the screw to the bone 12. This rotation can be imparted by a manual or active driver that engages the fastener assembly driver 24. The screw can be any type of screw for facilitating a secure attachment to the bone. The screw can include any type of thread pitch or thread count. When the fastener 22 is a bone pin or a nail, the fastener assembly driver 24 may receive axial impact forces to axially drive the bone pin to the bone. A tool, such as an impact driver, can be utilized to provide this axial force. When the fastener 22 is a bone clamp or a bone claw, the bone clamp or bone claw may be affixed to a portion of the bone. In the instance of FIG. 3, the fastener assembly driver 24 is fixed to the fastener 22. Advantageously, the tracker mount 10 utilizes a single fastener 22 thereby reducing the trauma to the bone.

As shown, the fastener assembly driver 24 includes first threads 25. Additionally, the fastener assembly 16 includes a first keying feature 26. As will be described in greater detail below, the first threads 25 of the fastener assembly driver 24 and the first keying feature 26 interface with the sleeve assembly. The fastener assembly driver 24 and the fastener 22 are both coupled to the first keying feature 26. The first keying feature 26 is located between the fastener 22 and the fastener assembly driver 24 along the axis AX1 of the fastener assembly 16.

In the instance of FIG. 3, the fastener assembly driver 24 is permanently fixed to the fastener 22 as the fastener assembly driver 24 remains fixed to the fastener 22 after the fastener 22 has been attached to the bone 12. In some instances, the fastener assembly driver 24 may be integrally formed with the first keying feature 26 and the fastener 22. In other instances, the fastener assembly driver 24 may be coupled to the fastener 22 to facilitate attaching of the fastener 22 to the bone and can be disconnected from the fastener 22 after the fastener 22 has been attached to the bone.

The fastener assembly 16 may include a stop 28 located above the fastener 22 for limiting driving of the fastener 22 beyond a predetermined distance (d). In the instance of FIG. 3, the predetermined distance (d) is illustrated as a length of the fastener 22 along the axis AX1. As the fastener 22 is attached to the bone 12, the stop 28 contacts the bone surface to limit driving of the fastener 22 beyond the predetermined distance (d). In some instances, the stop 28 may be integrally formed with a component of the fastener assembly 16. For example, the stop 28 may be integrally formed with the first keying feature 26 and/or the fastener 22. In some instances, the stop 28 may be removably coupled to a component of the fastener assembly 16. For example, the stop 28 may be coupled to the first keying feature 26 and to the fastener 22. The bottom surface of the keying feature 26 may also function as the stop 28 In some instances, the stop 28 may be optionally omitted from the fastener assembly 16.

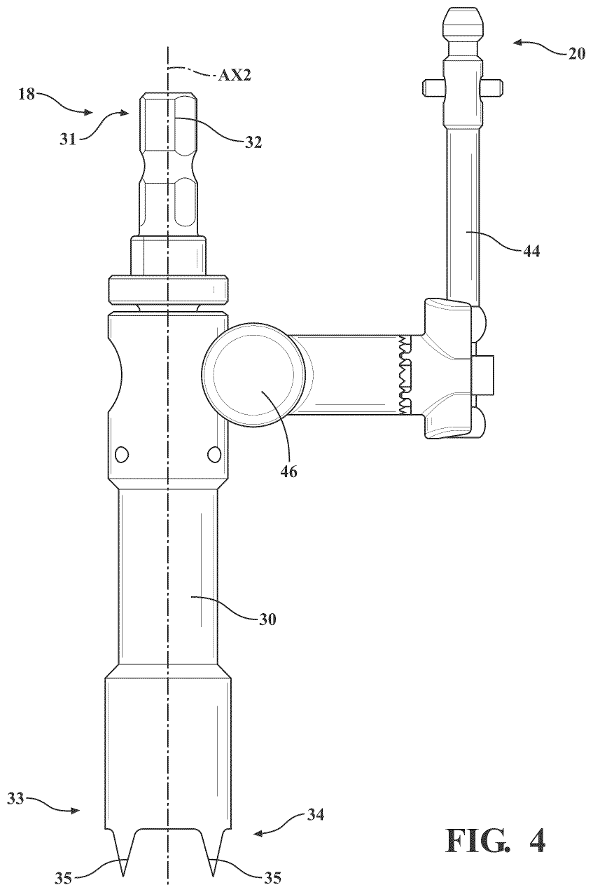

The sleeve assembly 18 is further shown in FIG. 4. As shown, the sleeve assembly 18 extends between a proximal part 31 and a distal part 33 along an axis AX2. The sleeve assembly 18 includes a sleeve 30, a sleeve assembly driver 32, and one or more engagement features 35 located at the distal part 33.

Referring to FIG. 1, the one or more engagement features 35 are configured to engage a surface of the bone 12, such as the femur (F), to mount the sleeve 30 to the bone. In the instance of FIG. 4, the one or more engagement features 35 include one or more teeth integrally formed with the sleeve 30 and configured to engage a surface of the bone. The one or more engagement features 35 may include any suitable shape and/or size for engaging a surface of the bone to mount the tracker mount 10 to the bone. For example, the engagement features 35 can take the form of a crown with several circumferential spikes or projections.

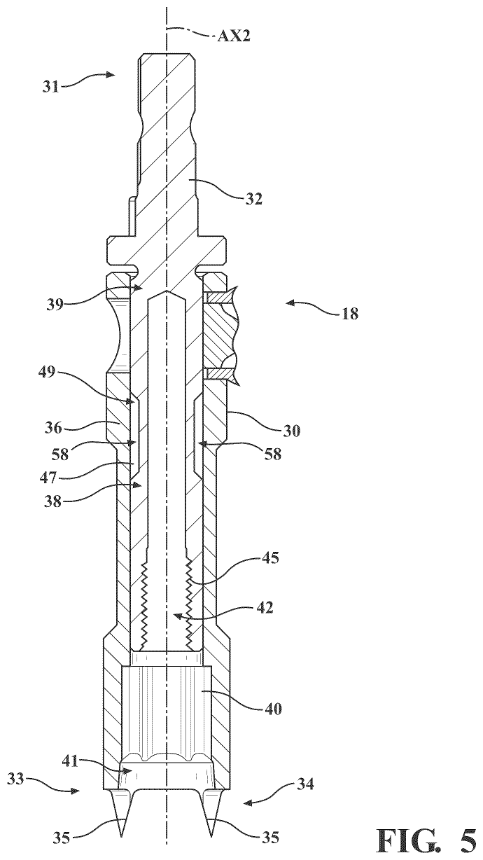

The sleeve 30 is further shown in FIG. 5 and the sleeve 30 includes a body 36, which defines a channel 38 including a first end 39 and a second end 41. The channel 38 includes a second keying feature 40. As will be described in greater detail below, the second keying feature 40 is configured to interface with the first keying feature 26 of the fastener assembly 16 to prevent rotation of the sleeve 30 relative to the fastener assembly 16 during mounting of the tracker mount 10.

The sleeve assembly driver 32 is further shown in FIG. 5. The sleeve assembly driver 32 is disposed, or adapted to be disposed, within the channel 38 of the sleeve 30. In one example, the sleeve assembly driver 32 is axially locked relative to the sleeve 30, but freely rotatable with respect to the sleeve 30. In another example, the sleeve assembly driver 32 can be freely inserted into the sleeve 30 as a separate component. The sleeve assembly driver 32 is adapted to couple to the fastener assembly 16. As will be explained in greater detail below, the sleeve assembly driver 32 is configured to couple with the fastener assembly driver 24 of the fastener assembly 16. Specifically, the sleeve assembly driver 32 is configured to be rotated to couple to the fastener assembly driver 24 to force the engagement features 35 into the bone. As the sleeve assembly driver 32 moves toward the bone, the one or more engagement features 35 correspondingly move into the bone according to a displacement of the sleeve assembly driver 32.

As shown in FIG. 5, the sleeve assembly driver 32 may define a second channel 42 and the sleeve assembly driver 32 includes second threads 45 located in the second channel 42. The second threads 45 are configured to interface with the first threads 25 of the fastener assembly driver 24 to couple the sleeve assembly driver 32 to the fastener assembly driver 24 in response to rotation of the sleeve assembly driver 32. In other words, the sleeve assembly driver 32 defines a female threaded configuration that receives a corresponding male threaded configuration of the fastener assembly driver 24. However, the opposite is contemplated. The sleeve assembly driver 32 may define a male threaded configuration that is threaded into a corresponding female threaded configuration of the fastener assembly driver 24. In such instances, fastener assembly driver 24 defines the second channel 42, rather than the sleeve assembly driver 32.

The sleeve assembly driver 32 is accessible through the second end 41 of channel, including through the second keying feature 40 portion of the channel 38. The sleeve assembly driver 32 extends through the first end 39 of the channel 38. The sleeve assembly driver 32 can be placed into the sleeve 30 through the first end 39 of the channel 38. Alternatively, the sleeve assembly driver 32 can remain axially constrained to the body 36 of the sleeve 30 such that the sleeve assembly driver 32 extends through the first end 39 of the channel 38 in a constrained location.

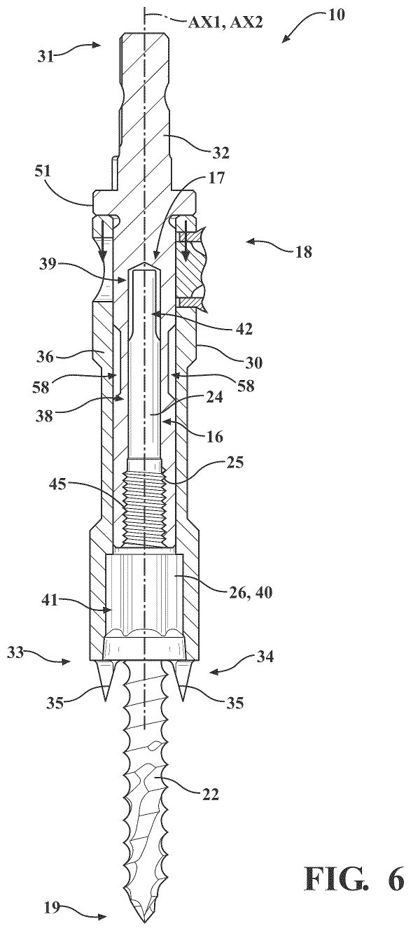

In some implementations, such as the implementation shown in FIG. 6, the sleeve assembly driver 32 may be freely inserted into the sleeve channel 38 from the first end 39 of the channel 38 (i.e., without trapping the sleeve assembly driver 32 within the sleeve). The implementation enabling this function is shown in FIG. 6, where the sleeve assembly driver 32 and the channel 38 include smooth, uninterrupted surfaces, thereby allowing the sleeve assembly driver 32 to be freely inserted into or removed from the channel 38. In the instance of FIG. 6, the sleeve assembly driver 32 includes an abutment 51 configured to contact the top of the sleeve 30. As the sleeve assembly driver 32 is driven down towards the bone by engaging the fastener assembly 16, the sleeve assembly driver 32 will correspondingly force the sleeve 30 to move down once the abutment 51 contacts the top of the sleeve 30.

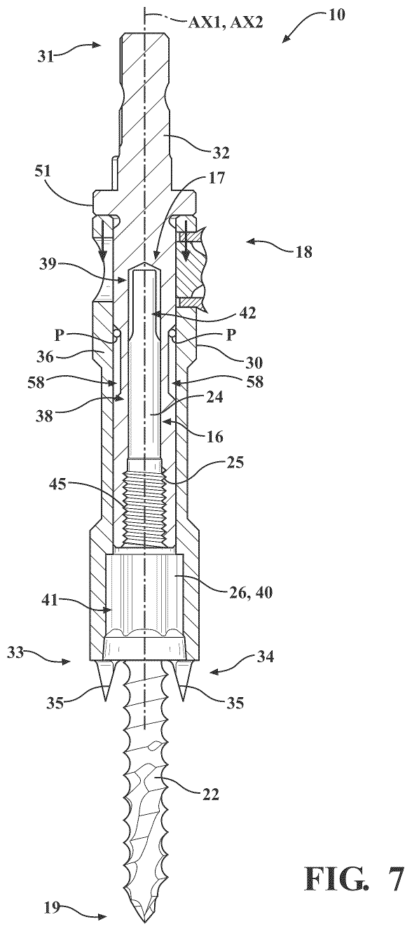

In some implementations, such as the implementation shown in FIG. 7, the sleeve assembly driver 32 and the sleeve 30 may be coupled and axially constrained by one or more pins to constrain movement of the sleeve assembly driver 32 relative to the sleeve 30 to a range. The implementation enabling this function is shown in FIG. 7, where the one or more pins P are shown as being disposed within a channel 58 defined between the sleeve assembly driver 32 and the sleeve 30. The one or more pins P may be affixed to the sleeve body 36 and extend through the channel 58 to constrain movement of the sleeve assembly driver 32 to an axial length corresponding to an axial length of the channel 58. The axial length of the channel 58 may be dimensioned to allow the sleeve assembly driver 32 to allow interfacing of the first threads 25 of the fastener assembly driver 24 and the second threads 45 of the sleeve assembly driver 32. Additionally, in the instance of FIG. 7, the sleeve assembly driver 32 includes the abutment 51 configured to contact the top of the sleeve 30. As such, the axial length of the channel 58 may be dimensioned to allow the abutment 51 to contact the top of the sleeve 30, and to allow the abutment 51 to force the sleeve 30 to toward the bone as the sleeve assembly driver 32 is driven towards the bone. In some instances, based on a size of the channel 58, the sleeve 30, and the sleeve assembly driver 32, the one or more pins P may additionally or alternatively force the sleeve 30 to toward the bone as the sleeve assembly driver 32 is driven towards the bone.

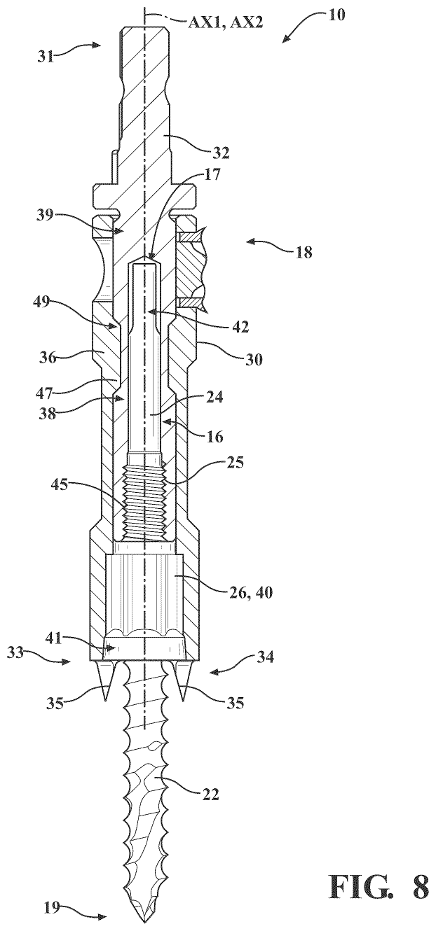

In some implementations, such as the implementation shown in FIG. 8, to facilitate axial constraint between the sleeve assembly driver 32 and the sleeve 30, one of these components may include a projection 47 and the other component may include a receiving portion 49 shaped to receive the projection 47. This configuration axially constraints the components while enabling unconstrained rotational movement of the sleeve assembly driver 32 relative to the sleeve 30. The sleeve assembly driver 32 is driven down towards the bone by engaging the fastener assembly 16 through rotational movement. Meanwhile, the sleeve 30 will correspondingly move down with the sleeve assembly driver 32 due to the bi-directional axial constraint therebetween. In the example shown, the sleeve 30 include the projection 47 and the sleeve assembly driver 32 includes the receiving portion 49 shaped to receive the projection 47. The projection 47 and the receiving portion 49 are located between the outer surface of the sleeve 30 and the second channel 42. The projection 47 and the receiving portion 49 may be radially or annularly formed to enable free rotational movement of the sleeve assembly driver 32 relative to the sleeve. The projection 47 shown in FIG. 8 includes a trapezoidal shape and the receiving portion 49 is shaped to receive the trapezoidal shape of the projection 47. The sloped sides of the trapezoidal shape provide a self-centering constraint on bi-directional axial movement of the sleeve assembly driver 32. In other instances, the projection 47 and the receiving portion 49 may include any suitable shape for axially constraining the sleeve assembly driver 32 to the sleeve body 36. In FIG. 8, the projection 47 is integrally formed with the sleeve body 36. However, the projection 47 may be a separate component that is coupled to the sleeve body 36.

As described, the sleeve assembly 18 is adapted to be coupled to the fastener assembly 16. As shown in FIG. 6, once the sleeve assembly 18 is coupled to the fastener assembly 16, the axis AX2 of the sleeve assembly 18 is aligned with the axis AX1 of the fastener assembly 16. The sleeve assembly 18 is disposed over the fastener assembly 16 such that the first keying feature 26 and the second keying feature 40 interface to prevent rotation of the sleeve 30 relative to the fastener assembly 16. The first keying feature 26 interfaces with the second keying feature 40 by fitting snugly within the second keying feature 40 to prevent movement relative to the second keying feature 40. Rotation of the sleeve 30 relative to the fastener assembly 16 is prevented by surface constraint between the first keying feature 26 relative to the second keying feature 40.

The first keying feature 26 and the second keying feature 40 may include any suitable shape or mechanism for preventing rotation of the sleeve 30 relative to the fastener assembly 16. For example, in the instances shown herein, the first keying feature 26 includes external polygonal shaped walls and the second keying feature 40 includes internal polygonal shaped walls, wherein the external polygonal shaped walls of the first keying feature 26 are configured to fit within the internal polygonal shaped walls of the second keying feature 40. The external and internal polygonal shaped walls may include any polygonal shape. For instance, the external and internal polygonal shaped walls may be external and internal hexagonal or octagonal shaped walls. As another example, the first and second keying features 26, 40 may include a locking mechanism for preventing movement of the first keying feature 26 relative to the second keying feature 40. For example, one keying feature 26, 40 may have a pin or projection and the other keying feature 26, 40 may have a slot or groove to receive the pin/projection to thereby lock rotational movement.

Additionally, it is contemplated that the male/female configuration of the first keying feature 26 and the second keying feature 40 are inverted relative to what is shown. In other words, the keying feature 26 of the fastener assembly 16 may be a channel and the second keying feature 40 of the sleeve assembly 18 may be a male configuration configured to be inserted into the first keying feature 26.

As shown in FIG. 6, the sleeve assembly 18 is disposed over the fastener assembly 16 such that the second channel 42 of the sleeve assembly driver 32 receives the fastener assembly driver 24 of the fastener assembly 16. The first threads 25 of the fastener assembly driver 24 and the second threads 45 of the sleeve assembly driver 32 are configured to interface in response to rotation of the sleeve assembly driver 32. In this way, rotation of the sleeve assembly driver 32 couples the sleeve assembly driver 32 to the fastener assembly driver 24 of the fastener assembly 16.

The fastener assembly driver 24 and the second channel 42 may include any suitable shape and size such that second channel 42 may receive the fastener assembly driver 24. For example, the fastener assembly driver 24 is minimized to allow the sleeve assembly 18 to be disposed over the fastener assembly 16. Additionally, other components of the sleeve assembly 18 and the fastener assembly 16 may include any size and shape to allow the sleeve assembly 18 to be disposed over the fastener assembly 16. In some instances, a size of components of the sleeve assembly 18 and components of the fastener assembly 16 may be optimized to enable the tracker mount 10 to reliably support a weight of a tracker attached to the tracker mount 10, while also minimizing a size of the fastener 22 required for stably mounting the tracker mount 10.

Additionally, the sleeve assembly 18 is adapted to be disposed over the fastener assembly 16 after the fastener assembly 16 has been attached to the bone 12. As such, rotation of the sleeve assembly driver 32 couples the sleeve assembly driver 32 to the fastener assembly driver 24 of the fastener assembly 16, moves the sleeve assembly driver 32 toward the bone, and moves the one or more engagement features 35 into the bone.

In the instances shown herein, the second channel 42 is configured to receive the fastener assembly driver 24 when the sleeve assembly 18 is disposed over the fastener assembly 16. Accordingly, the first threads 25 of the fastener assembly driver 24 are shown in FIG. 6 to be external threads and the second threads 45 of the sleeve assembly driver 32 are shown to be corresponding internal threads to allow the first threads 25 and the second threads 45 to interface with one another in response to rotation of the sleeve assembly driver 32. However, in other instances, the fastener assembly driver 24 may instead include a channel configured to receive the sleeve assembly driver 32 when the sleeve assembly 18 is disposed over the fastener assembly 16. In such instances, the sleeve assembly driver 32 may omit the second channel 42. Furthermore, in such instances, the threads of the sleeve assembly driver 32 may be external threads and the threads of the fastener assembly driver 24 may be corresponding internal threads. Additionally, in instances where the fastener assembly driver 24 is received by the second channel 42, a clearance may be provided between the distal part 17 of the fastener assembly driver 24 and the second channel 42 to provide unobstructed rotation between the components and to prevent damaging of the fastener assembly driver 24 and the sleeve assembly driver 32. Similarly, in instances where the fastener assembly driver 24 may instead include a channel configured to receive the sleeve assembly driver 32, a clearance may be provided between the channel of the fastener assembly driver 24 and the sleeve assembly driver 32 to provide unobstructed rotation between the components and to prevent damaging of the fastener assembly driver 24 and the sleeve assembly driver 32.

The tracker interface 20 is configured to be coupled to the sleeve assembly 18. The tracker interface 20 may be removably attached to the sleeve assembly 18 or permanently fixed thereto. In one example, referring to FIG. 2, the tracker interface 20 is coupled to the sleeve assembly 18 via a support arm 44. As shown, the support arm 44 is coupled to the sleeve assembly 18, and the tracker interface 20 is coupled to the support arm 44. In some instances, such as the instance shown herein, the support arm 44 is an adjustable support arm 44. The adjustable support arm 44 may be adjustable such that the tracker interface 20 may be positioned in a variety of positions and/or orientations. The adjustable support arm 44 may include any suitable interface to enable the adjustable support arm 44 to be adjusted into a variety of positions and/orientations. For example, in FIG. 2, the adjustable support arm 44 includes a push button 46, wherein depression of the push button 46 enables the adjustable support arm 44 to rotate about an axis defined through the push button. In other instances, the adjustable support arm 44 may be adjusted by the tightening and loosening of a fastener, such as a screw. Additionally, in alternative instances, the support arm 44 may be rigid and not adjustable. In yet another example, the tracker interface 20 may provide a coupling directly on the sleeve assembly 18 without any support arm.

The tracker interface 20 is configured to attach to a tracker 48. Referring to the instance of FIG. 2, the tracker 48 is attached to the tracker interface 20. In the instance of FIG. 4, the tracker 48 is removed and no longer attached to the tracker interface 20. The tracker 48 may be removably attached to the tracker interface 20 using any suitable means. For example, the tracker 48 or the tracker interface 20 may include a receptacle shaped to receive a portion of the other of the tracker 48 and the tracker interface 20. In such an instance, the receptacle may receive the portion of the other of the tracker 48 and the tracker interface 20 to attach the tracker 48 to the tracker interface 20. An example of the mounting between the tracker 48 and the tracker interface 20 can be like that described in U.S. Pat. No. 10,537,395, entitled “Navigation Tracker with Kinematic Connector Assembly”, the entire contents of which are hereby incorporated by reference.

### III. Method of Mounting the Tracker Mount

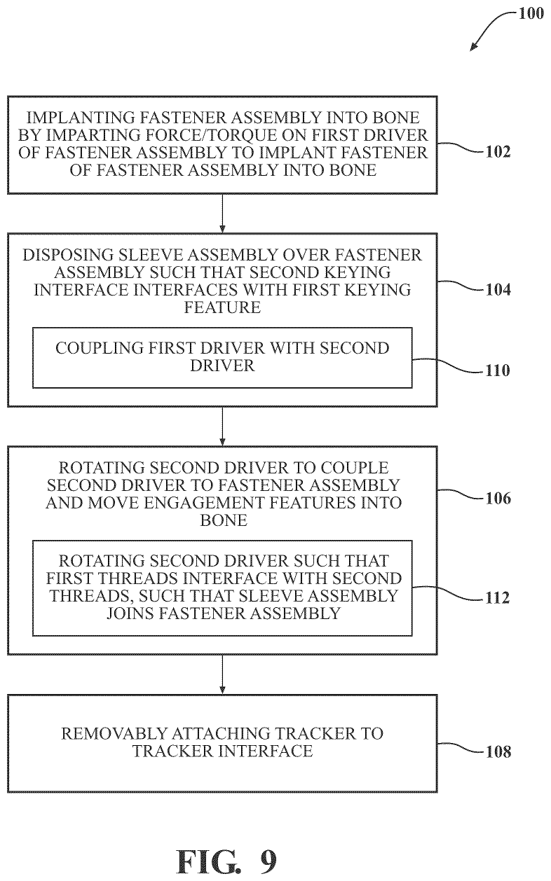

FIG. 9 illustrates an example method 100 of mounting the tracker mount 10 to the bone. As shown, the method 100 includes a step 102 of imparting a force/torque to the fastener assembly driver 24 of the fastener assembly 16 to attach the fastener 22 of the fastener assembly 16 to the bone; a step 104 of disposing the sleeve assembly 18 over the fastener assembly 16 such that the second keying feature 40 interfaces with the first keying feature 26 to prevent rotation of the sleeve 30 relative to the fastener assembly 16; a step 106 of rotating the sleeve assembly driver 32 to couple the sleeve assembly driver 32 to the fastener assembly 16 and move the one or more engagement features 35 into the bone; and optionally, a step 108 of removably attaching the tracker 48 to the tracker interface 20.

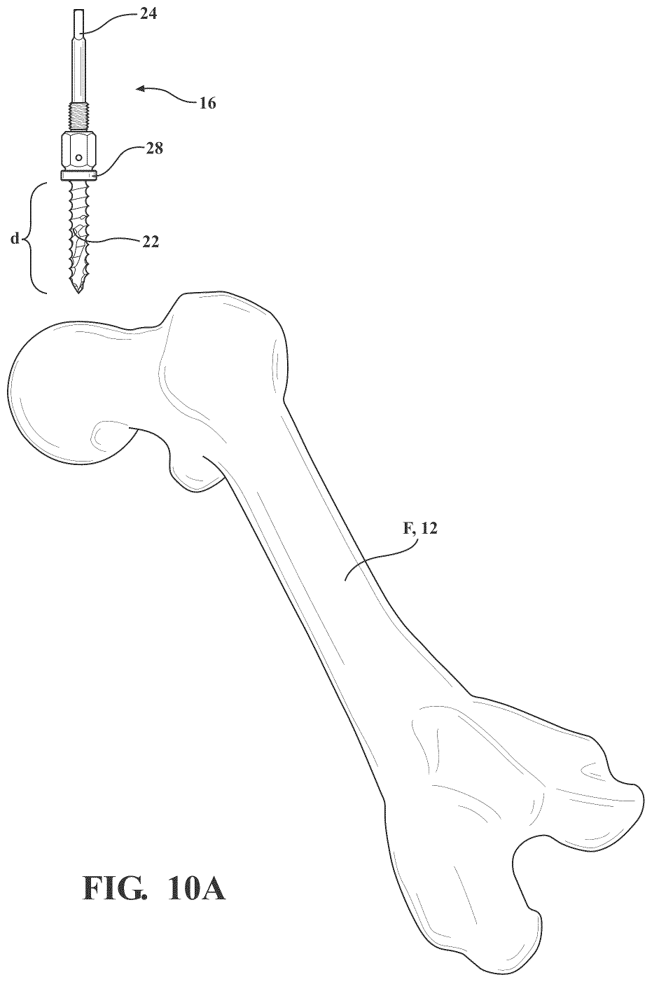

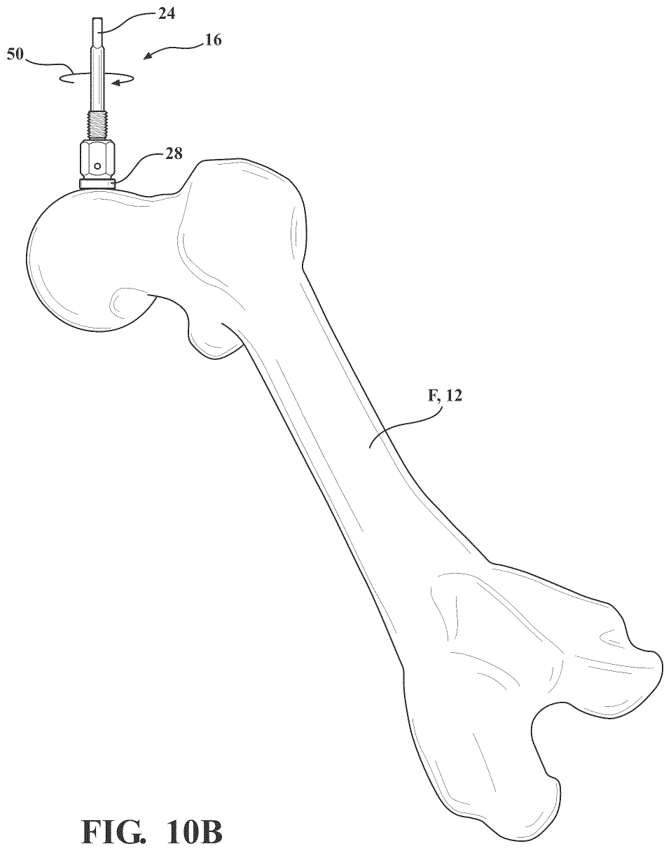

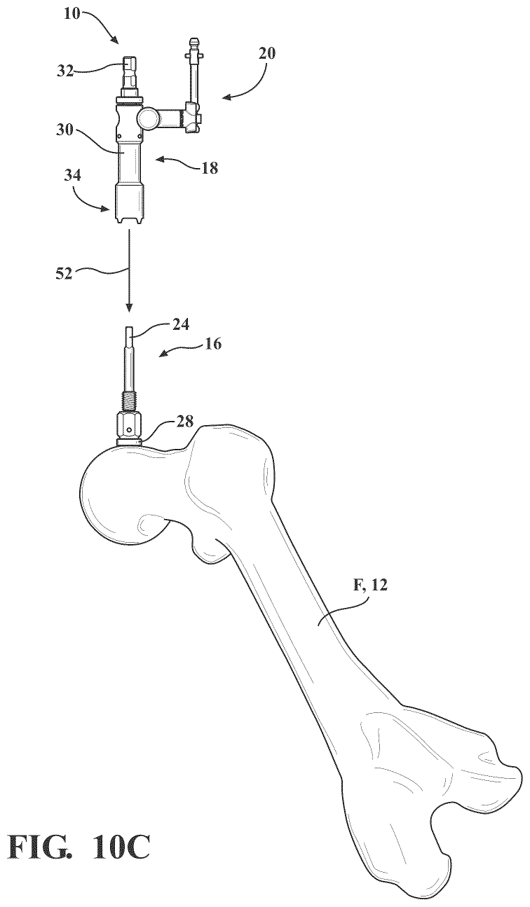

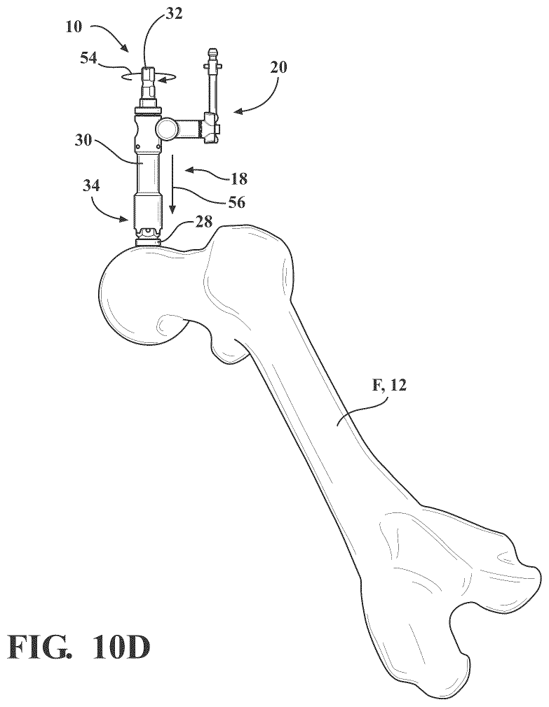

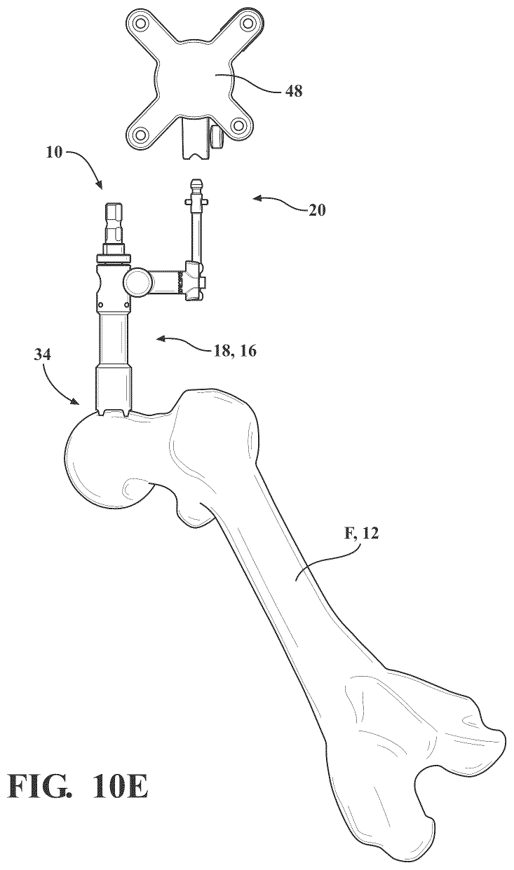

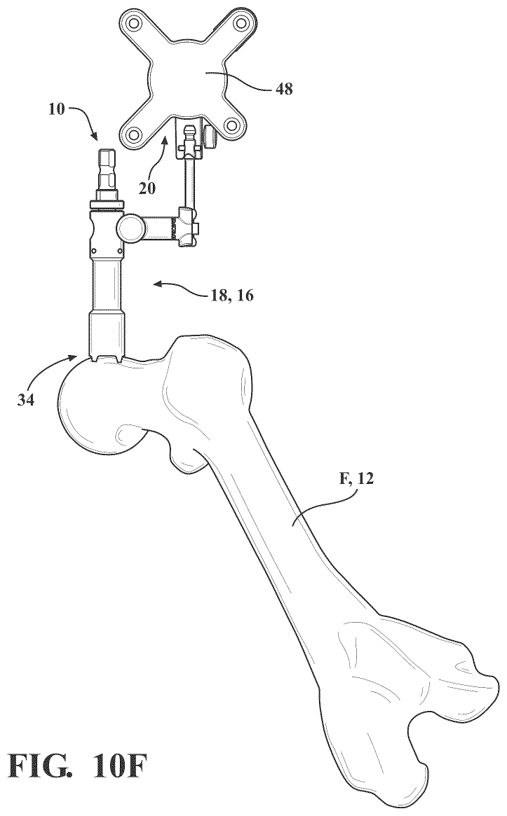

FIGS. 10A and 10B further illustrate the step 102 of attaching the fastener assembly 16 to the bone. As shown in FIGS. 10A and 10B, the fastener 22 is attached to the bone 12, which is illustrated as a femur (F). When the fastener 22 is a screw, the fastener assembly driver 24 is adapted to be rotated, as indicated by arrow 50 (FIG. 10B), to attach the fastener 22 to the bone. Again, this can be performed using a manual or powered driver that engages the fastener assembly driver 24. If a bone pin is utilized, the fastener assembly driver 24 may be attached to a tool (e.g., impact driver) configured to impart an axial driving force to the fastener assembly driver 24 to attach the pin to the bone. In either instance, a pilot hole may first be formed into the bone 12 to prevent damaging the bone. As described, the fastener assembly 16 may include a stop 28 located above the fastener 22. As shown, in FIG. 10B, the stop 28 may be configured to contact the surface of the bone to limit driving of the fastener 22 beyond the predetermined distance (d) (shown in FIG. 10A). The stop 28 thus provides haptic feedback to the user indicating when force/torque should no longer be imparted.

FIG. 10C illustrates the step 104 of disposing the sleeve assembly 18 over the fastener assembly 16. The step 104 occurs after the fastener assembly 16 has been attached during step 102 (FIG. 10B). As indicated by arrow 52 in FIG. 10C, the sleeve assembly 18 is disposed over the fastener assembly 16. The sleeve assembly 18 is disposed over the fastener assembly 16 such that the axis AX1 of the fastener assembly 16 and the axis AX2 of the sleeve assembly 18 are aligned (as shown in FIG. 6). As shown, the sleeve assembly 18 is disposed over the fastener assembly 16 such that the first keying feature 26 and the second keying feature 40 interface to prevent rotation of the sleeve 30 relative to the fastener assembly 16. Furthermore, the keying advantageously enables the sleeve assembly 18 to be held in place should the user need to temporarily release the sleeve assembly 18 to perform other steps. This keying feature maintains alignment of the sleeve assembly 18 relative to the fastener assembly 16 and avoids the need to have the user continually hold the sleeve assembly 18 in place and/or maintain alignment thereof.

The step 104 of disposing the sleeve assembly 18 over the fastener assembly 16 may optionally include a step 110 of coupling the fastener assembly driver 24 with the sleeve assembly driver 32. Referring to FIG. 6, the sleeve assembly 18 is disposed over the fastener assembly 16 such that the second channel 42 of the sleeve assembly driver 32 receives the fastener assembly driver 24. However, in an alternative implementation, the fastener assembly driver 24 may instead include a channel configured to receive the sleeve assembly driver 32. In such instances, step 110 may instead include a step of receiving the sleeve assembly driver 32 with the fastener assembly driver 24.

FIG. 10D illustrates the step 106 of rotating the sleeve assembly driver 32 to couple the sleeve assembly driver 32 to the fastener assembly 16, wherein rotation of the sleeve assembly driver 32 is indicated by arrow 54. Rotating the sleeve assembly driver 32 may be performed using any tool, such as a manual or powered rotary driver. Step 106 may include a step 112 of rotating the sleeve assembly driver 32 such that the first threads 25 of the fastener assembly driver 24 interface with the second threads 45 of the second channel 42. In this way, as the first threads 25 interface with the second threads 45, the sleeve assembly driver 32 joins the fastener assembly driver 24, and the sleeve assembly 18 joins the fastener assembly 16. As described, the threading may be male/female or female/male, respectively.

During step 112, the first threads 25 of the fastener assembly driver 24 interface with the second threads 45 of the second channel 42 to drive the one or more engagement features 35 into the bone. More specifically, the first threads 25 of the fastener assembly driver 24 interface with the second threads 45 of the second channel 42 to move the sleeve assembly driver 32 toward the bone. Movement of the sleeve assembly driver 32 causes movement of the sleeve 30 toward the bone, as indicated by arrow 56. Movement of the sleeve 30 toward the bone causes the one or more engagement features 35 to move into the bone 12. Since the fastener assembly 16 is fixed to the bone, the fastener assembly 16 provides a counter-acting force against the force imparted by the sleeve assembly driver 32 and to thereby force the engagement features 35 to into the bone 12.

As described, there are several implementations by which the sleeve assembly driver 32 interacts with the sleeve 30. In the implementation of FIGS. 6 and 7, movement of the sleeve assembly driver 32 causes movement of the abutment 51 relative to the sleeve 30. Once movement of the sleeve assembly driver 32 causes the abutment 51 to contact the sleeve 30, additional movement of the sleeve assembly driver 32 causes the abutment 51 to force the sleeve 30 and engagement features 35 to move toward the bone. In the implementation of FIG. 8, movement of the sleeve assembly driver 32 causes movement of the sleeve 30 toward the bone because the body 36 of the sleeve 30 is axially constrained to the sleeve assembly driver 32 through the receiving portion 49 and the projection 47. In this example, as the sleeve assembly driver 32 moves toward the bone 12, the axial constraint causes the sleeve 30 and engagement features 35 to correspondingly move toward the bone.

During step 106, the first and second keying features 25, 40 can prevent rotation of the sleeve 30 relative to the fastener assembly 16 before, during or after rotation of the sleeve assembly driver 32. During rotation of the sleeve assembly driver 32, the first and second keying features 25, 40 are rotationally constrained relative to one another but the features are can axially move relative to one another. In this way, as the sleeve assembly driver 32 rotates and moves toward the bone, the sleeve 30 and engagement features 35 moves toward the bone without rotating. Advantageously, by moving into the bone without rotating, the one or more engagement features 35 are prevented from unnecessarily damaging the bone or skiving off the surface of the bone. As an additional advantage, because the first and second keying features 25, 40 prevent rotation of the sleeve 30, the first and second keying features 25, 40 provide rotational stability before, during, and after mounting the tracker mount 10. The increased rotational stability provides for increased durability of the components of the tracker mount 10 which, in turn, allows for reuse of the components of the tracker mount 10. The rotationally stability further helps ensure a stable tracker mounting for tracking accuracy and resists dislodging of the mount 10 due to collisions.

FIGS. 10E and 10F illustrate the step 108 of removably attaching the tracker 48 to the tracker interface 20. Although FIG. 9 illustrates step 108 as occurring after steps 102, 104, 106, 110, and 112, step 108 may occur prior to or after any of the steps 102, 104, 106, 110, 112. For example, the step 208 of removably attaching the tracker 48 to the tracker interface 20 may occur after the step 106 of rotating the sleeve assembly driver 32 to couple the sleeve assembly driver 32 to the fastener assembly 16 and move the one or more engagement features 35 into the bone. Such an example is shown in FIGS. 10E and 10F, where the tracker 48 is attached to the tracker interface 20 after the engagement features 35 are moved into the bone. As another example, the step 208 of removably attaching the tracker 48 to the tracker interface 20 may occur prior to the step 106 of rotating the sleeve assembly driver 32 to couple the sleeve assembly driver 32 to the fastener assembly 16 and move the one or more engagement features 35 into the bone.

The described tracker mount 10 resolves numerous problems associated with conventional mounts. For example, the described tracker mount 10 utilizes a single fastener thereby minimizing potential damage to the bone 12 and reducing the footprint of the mount 10. Furthermore, the described tracker mount 10 ensures rotational stability, thereby reducing the likelihood that the tracker mount 10, or tracking device, become loose relative to the bone. Such stability increases the localizer's ability to accurately track the bone. The described tracker mount 10 is also durable and robust and hence, can be sterilized and utilized repeatedly thereby reducing costs. The durability also enhances stability of the design. Moreover, the described tracker mount 10 is ergonomically optimized. For example, the user attaches the fastener assembly to the bone first, without the sleeve attached thereto. By separating these parts, the user can drive the fastener without obstructed visibility by the sleeve. Other advantages not specifically described herein will be recognized from the detailed description and drawings.

The above tracker mount 10 can be designed with a configuration, look, or function that differs from the implementation specifically shown in the Figures. Other configurations may include equivalents to any components described herein which operate with a similar function and accomplish a similar result.

Those having ordinary skill in the art will appreciate that aspects of the embodiments described and illustrated herein can be interchanged or otherwise combined. It will be further appreciated that the terms “include,” “includes,” and “including” have the same meaning as the terms “comprise,” “comprises,” and “comprising.” Moreover, it will be appreciated that terms such as “first,” “second,” “third,” and the like are used herein to differentiate certain structural features and components for the non-limiting, illustrative purposes of clarity and consistency.

Several configurations have been discussed in the foregoing description. However, the configurations discussed herein are not intended to be exhaustive or limit the invention to any particular form. The terminology which has been used is intended to be in the nature of words of description rather than of limitation. Many modifications and variations are possible in light of the above teachings and the invention may be practiced otherwise than as specifically described.

What is claimed is:

1. A tracker mount comprising:
a fastener assembly comprising:
a fastener adapted to be attached to a bone; and
a first keying feature; and
a sleeve assembly adapted to be disposed over the fastener assembly and comprising:
a sleeve comprising:
a body defining a channel, wherein the channel includes a second keying feature to interface with the first keying feature to prevent rotation of the sleeve relative to the fastener assembly; and
an engagement feature at a distal part of the sleeve to engage a surface of the bone; and
a sleeve assembly driver at least partially disposed within the channel, the sleeve assembly driver being adapted to be rotated to couple to the fastener assembly and move the engagement feature into the bone; and
a tracker interface coupled to the sleeve assembly and configured to removably attach to a tracker.

2. The tracker mount of claim 1, wherein the fastener assembly further comprises a fastener assembly driver coupled to the fastener and adapted to receive force/torque to attach the fastener to the bone.

3. The tracker mount of claim 2, wherein the sleeve assembly driver is configured to be rotated to couple to the fastener assembly driver of the fastener assembly.

4. The tracker mount of claim 3, wherein the sleeve assembly driver defines a second channel configured to receive the fastener assembly driver, and wherein the fastener assembly driver further comprises first threads and the sleeve assembly driver further comprises second threads located in the second channel to interface with the first threads in response to rotation of the sleeve assembly driver.

5. The tracker mount of claim 4, wherein the sleeve assembly driver includes an abutment configured to contact the sleeve such that rotation of the sleeve assembly driver causes the abutment to move the sleeve to move the engagement feature into the bone.

6. The tracker mount of claim 1, wherein the body of the sleeve includes a projection and the sleeve assembly driver includes a receiving portion shaped to receive the projection to axially constrain the body of the sleeve to the sleeve assembly driver such that rotation of the sleeve assembly driver moves the sleeve to move the engagement feature into the bone.

7. The tracker mount of claim 2, wherein the fastener assembly driver of the fastener assembly is permanently fixed to the fastener.

8. The tracker mount of claim 1, wherein the sleeve assembly is adapted to be disposed over the fastener assembly after the fastener of the fastener assembly is attached to the bone.

9. The tracker mount of claim 1, wherein the first keying feature of the fastener assembly includes external polygonal shaped walls, and wherein the second keying feature of the sleeve assembly includes internal polygonal shaped walls.

10. The tracker mount of claim 2, where the first keying feature is located between the fastener and the fastener assembly driver.

11. The tracker mount of claim 1, wherein the channel of the sleeve includes a first end and a second end, wherein the sleeve assembly driver extends through the first end, and wherein the second keying feature is located at the second end.

12. The tracker mount of claim 1, wherein the engagement feature comprises one or more teeth integrally formed with the sleeve and being configured to engage the surface of the bone.

13. The tracker mount of claim 1, wherein a support arm is coupled to the sleeve assembly and the tracker interface is coupled to the support arm.

14. The tracker mount of claim 1, wherein the fastener assembly further comprises a stop located above the fastener for limiting driving of the fastener beyond a predetermined distance.

15. The tracker mount of claim 14, wherein the stop is configured to contact the bone to limit driving of the fastener beyond the predetermined distance.

16. The tracker mount of claim 1, wherein the fastener comprises one of: a screw, a bone pin, or a nail.

17. A method of mounting a tracker mount to a bone, the tracker mount including a fastener assembly, a sleeve assembly, and a tracker interface coupled to the sleeve assembly, the fastener assembly including a fastener and a first keying feature, the sleeve assembly including a sleeve and a sleeve assembly driver, the sleeve including an engagement feature at a distal part of the sleeve and a body defining a channel including a second keying feature, the method comprising steps of:
attaching the fastener of the fastener assembly to the bone;
after attaching the fastener, disposing the sleeve assembly over the fastener assembly such that the second keying feature interfaces with the first keying feature for preventing rotation of the sleeve relative to the fastener assembly; and
rotating the sleeve assembly driver to couple the sleeve assembly driver to the fastener assembly for moving the engagement feature into the bone.

18. The method of claim 17, wherein the fastener assembly further includes a fastener assembly driver coupled to the fastener, the fastener assembly driver including first threads, wherein the sleeve assembly driver defines a second channel and further includes second threads located in the second channel, and wherein the method further comprises steps of:
imparting a force/torque to the fastener assembly driver to attach the fastener of the fastener assembly to the bone;
receiving the fastener assembly driver with the sleeve assembly driver; and
rotating the sleeve assembly driver for causing the first threads to interface with the second threads and for causing the sleeve assembly to join with the fastener assembly and for causing the engagement feature to move into the bone.

19. The method of claim 17, further comprising a step of removably attaching a tracker to the tracker interface.

20. The method of claim 19, wherein the step of removably attaching a tracker to the tracker interface occurs prior to the step of rotating the sleeve assembly driver to couple the sleeve assembly driver to the fastener assembly and move the engagement feature into the bone.

21. The method of claim 19, wherein the step of removably attaching a tracker to the tracker interface occurs after to the step of rotating the sleeve assembly driver to couple the sleeve assembly driver to the fastener assembly and move the engagement feature into the bone.

22. A bone mount comprising:
a fastener assembly comprising: a fastener adapted to be attached to a bone and a first keying feature;
a sleeve assembly adapted to be disposed over the fastener assembly and comprising: a sleeve comprising a body defining a channel, wherein the channel includes a second keying feature to interface with the first keying feature to prevent rotation of the sleeve relative to the fastener assembly; and an engagement feature at a distal part of the sleeve to engage a surface of the bone.

23. A tracker mount comprising:
a fastener assembly comprising:
a fastener adapted to be attached to a bone; and
a first keying feature; and
a sleeve assembly adapted to be disposed over the fastener assembly and comprising:
a sleeve comprising:
a body defining a channel, wherein the channel includes a second keying feature to interface with the first keying feature to prevent rotation of the sleeve relative to the fastener assembly; and
an engagement feature at a distal part of the sleeve to engage a surface of the bone; and
a driver at least partially disposed within the channel, the driver being adapted to be rotated to couple to the fastener assembly and move the engagement feature into the bone; and
a tracker interface coupled to the sleeve assembly and configured to removably attach to a tracker.

24. The tracker mount of claim 23, wherein the fastener comprises a bone clamp or a bone claw.
